SEQUENCE LISTING (3 sequences)

SEQ ID NO: 1 - protein, 15 aa, Artificial Sequence
  human beta-defensin derived peptide(BD3-3)
GKCSTRGRKCCRRKK

SEQ ID NO: 2 - protein, 15 aa, Artificial Sequence
  human beta-defensin derived peptide(BD3-3-M1)
GKCSTRGRKCMRRKK

SEQ ID NO: 3 - protein, 15 aa, Artificial Sequence
  human beta-defensin derived peptide(BD3-3-M2)
GKCSTRGRKMCRRKK

(19) United States
(12) Patent Application Publication
(10) Pub. No.: US 2021/0138111 A1
(43) Pub. Date: May 13, 2021

(54) INTEGRAL BIOMATERIAL FOR REGENERATION OF BONE TISSUE AND FABRICATION METHOD THEREFOR

(71) Applicants: NIBEC CO., LTD., Chungcheongbuk-do (KR); SEOUL NATIONAL UNIVERSITY R&DB FOUNDATION, Seoul (KR)

(72) Inventors: Yoon Jeong PARK, Seoul (KR); Chong-Pyoung CHUNG, Seoul (KR); Jue-Yeon LEE, Gyeonggi- do (KR); Ju Yeon CHAE, Seoul (KR)

(21) Appl. No.: 16/623,167

(22) PCT Filed: Jul. 27, 2017
(86) PCT No.: PCT/KR2017/008112

§ 371 Date: Dec. 16, 2019

(30) Foreign Application Priority Data
Jun. 19, 2017 (KR) 10-2017-0077400

(51) Int. Cl. A61L 27/24; A61L 27/54; A61L 27/36

(52) U.S. Cl. CPC A61L 27/24; A61L 27/54; A61L 2300/41; A61L 2430/02; A61L 2300/404; A61L 27/3608

(57) ABSTRACT

The present invention relates to an integrated biomaterial for bone tissue regeneration and a method of preparing the same, and more particularly to an integrated biomaterial for bone tissue regeneration, which includes a lower structure consisting of an extracellular matrix protein and a bone mineral and an upper layer consisting of an extracellular matrix protein. In the integrated biomaterial for bone tissue regeneration according to the present invention, the lower structure consisting of an extracellular matrix protein and a bone mineral component realizes a natural bone tissue environment, and thus facilitates the regeneration of new bone, and particularly, the upper layer consisting of an extracellular matrix protein is placed thereon at an appropriate ratio, and thus not only prevents the infiltration of epithelial tissue or connective tissue but also maximizes bone tissue regeneration capability.

### TECHNICAL FIELD

The present invention relates to an integrated biomaterial for bone tissue regeneration and a method of preparing the same, and more particularly to an integrated biomaterial for bone tissue regeneration including a lower structure consisting of an extracellular matrix protein and a bone mineral and an upper layer consisting of an extracellular matrix protein, and a method of preparing the same.

### BACKGROUND ART

The periodontal tissue supporting the teeth broadly consists of alveolar bone, connective tissue constituting the periodontal membrane between the alveolar bone and the tooth, epithelial tissue and periodontal ligament tissue. The loss of alveolar bone due to the progression of periodontitis is accompanied by loss of periodontal ligament tissues, and normal recovery of alveolar bone and periodontal ligament tissues is impossible due to overgrowth of connective tissues at the site of tissue loss after periodontal treatment. In addition, even when new bone is generated, the periodontal ligament tissue may not be normally differentiated, resulting in loss of dental function. Therefore, to address these problems, an artificial barrier membrane is used together with bone substitute grafting as an alveolar bone regeneration surgery. When a bone graft material is used alone, there is a drawback in that the bone graft material is difficult to maintain at a graft site, and thus tissue regeneration is induced using a method of filling a bone loss site with a bone graft material and placing a shielding (barrier) membrane thereon and suturing the membrane (see FIG. 1). When a bone graft material and a barrier membrane are used at the same time, the bone graft material implanted on a lower side and the barrier membrane on an upper side must be in close contact with each other to facilitate bone regeneration, thus increasing the success rate of bone grafting. In contrast, if there is a gap between the bone graft material and the barrier membrane, the success rate of bone regeneration is lowered. However, applying separate products of a bone graft material and a barrier membrane makes it impossible to achieve perfect close contact between the two products. Therefore, if an integrated product having both a bone graft material and a barrier membrane function is developed, the success rate of bone regeneration can be increased. It may be possible to simultaneously obtain osteoconductivity of the bone graft material and a connective tissue preventive effect of the barrier membrane in one product, and thus convenience of use may be enhanced (see FIGS. 2A and 2B).

Until recently, the most frequently used graft material for bone regeneration surgery includes an autogenous bone graft material, an allogenic bone graft material, a xenogenic bone graft material, and a synthetic bone graft material, but autogenous bone requires secondary surgery for bone collection so that it is difficult to obtain such bone, allogenic bone is problematic in the possibility of contamination with a disease, and synthetic bone has low biocompatibility with natural bone tissue, and thus xenogenic bone is widely used.

In the case of barrier membranes, it has been reported that barrier membranes have been used to induce regeneration of periodontal tissue effectively over the past 20 years (J. Gottlow, et al., J. Clin. Perio, 13, (1986) pp. 604˜616), and since then, research on tissue induction and regeneration has been carried out using various materials as barrier membranes. Recently, among biodegradable barrier membranes, barrier membranes made of collagen are the most widely used, but have limitations such as weak mechanical strength and incomplete close contact with a bone graft material.

Meanwhile, bones and teeth in the human body contain approximately 80% minerals and water and 20% organic matter, and 80% of organic matter consists of collagenous proteins and 20% thereof consists of non-collagenous proteins. These protein components not only contribute to maintaining the production of hard tissue and structural strength and elasticity, but also act as a matrix for inducing the adhesion of hard tissue-forming cells such as osteoblasts and for orienting inorganic ion components constituting hard tissue components (Anselme, Osteoblast adhesion on biomaterials, Biomaterials, 21 (7): 667-81, 2000).

Existing xenogenic bone-derived bone graft materials contain only mineral components, from which proteins are completely removed, and thus do not have the same structure as that of natural bone. To address this problem, a biomaterial to which proteins constituting bone tissue, such as collagen, are introduced is being developed. However, it is impossible to prepare a stable biomaterial by simply mixing collagen and a bone graft material, and existing biomaterial preparation methods have limitations in preparation of a biomaterial having a barrier membrane function.

Meanwhile, a method of separately preparing an upper layer and a lower structure and binding the upper layer and the lower structure via covalent bonding using a chemical crosslinking agent in the final product stage has been attempted, but the crosslinking reaction hardly occurs in a solid phase state so that sufficient bonding cannot be formed, and when a product is allowed to sufficiently absorb a physiological saline solution or be hydrated therewith for use, there is a problem such as separation between the upper layer and the lower structure. In addition, when a chemical crosslinking agent is used, there should be no chemical crosslinking agent remaining in the product, but if it remains, toxicity may be caused at the graft site, and thus the chemical crosslinking agent exhibits an adverse effect in terms of safety. In addition, when the lower structure and the upper layer are separately implanted, a space is generated between the two structures, and thus connective tissue may infiltrate not only from the upper side but also from the side surface thereinto, thereby interfering with a bone regeneration process proceeding from the lower side, resulting in significantly reduced bone regeneration efficiency.

Therefore, to address the above-described problems, there is a need to develop an integrated biomaterial which not only realizes a bone tissue environment, but also has a barrier membrane function capable of preventing infiltration of connective tissue.

Therefore, as a result of having made intensive efforts to address the above-described conventional problems, the inventors of the present invention developed an integrated biomaterial to which extracellular matrix and bone mineral components are organically bound so as to have a composition similar to that of bone tissues, and having a barrier membrane function, and confirmed that such an integrated biomaterial has an excellent effect on bone regeneration, thus completing the present invention.

### DISCLOSURE

### Technical Problem

Therefore, the present invention has been made in view of the above problems, and it is an object of the present invention to provide an integrated biomaterial for bone tissue regeneration which not only realizes a bone tissue environment, but also prevents infiltration of connective tissues to thereby exhibit maximized bone tissue regeneration capability, and a method of preparing the same.

### Technical Solution

In accordance with the present invention, the above and other objects can be accomplished by the provision of an integrated biomaterial for bone tissue regeneration, the integrated biomaterial comprising: a lower structure including an extracellular matrix protein and a bone mineral component; and an upper layer including an extracellular matrix protein.

In accordance with an aspect of the present invention, the above and other objects can be accomplished by the provision of a use of an integrated biomaterial for bone tissue regeneration, the integrated biomaterial comprising: a lower structure including an extracellular matrix protein and a bone mineral component; and an upper layer including an extracellular matrix protein.

In accordance with another aspect of the present invention, there is provided a bone tissue regeneration method including implanting, into an individual in need of bone tissue regeneration, an integrated biomaterial comprising: a lower structure including an extracellular matrix protein and a bone mineral component; and an upper layer including an extracellular matrix protein.

In accordance with a further aspect of the present invention, there is provided a method of preparing an integrated biomaterial for bone tissue regeneration comprising: a lower structure including an extracellular matrix protein and a bone mineral component; and an upper layer including an extracellular matrix protein, the method comprising: (a) molding a lower structure mixture including an extracellular matrix protein and bone mineral particles; (b) aligning a structure of the lower structure mixture including an extracellular matrix protein and bone mineral particles; (c) placing an upper layer including an extracellular matrix protein thereon; (d) binding the upper layer and the lower structure; (e) lyophilizing the resulting structure; and (f) thermally cross-linking the extracellular matrix protein of the upper layer.

### DESCRIPTION OF DRAWINGS

The above and other objects, features and other advantages of the present invention will be more clearly understood from the following detailed description taken in conjunction with the accompanying drawings, in which:

FIG. 1 is a schematic view illustrating a general bone graft procedure, wherein, after being first filled with a bone graft material, a barrier membrane is placed thereon and sutured;

FIG. 2A is a schematic view of an integrated biomaterial in which a bone graft material and a barrier membrane function are integrated, FIG. 2B is a differential scanning electron microscope image of an integrated biomaterial in which a bone graft material and a barrier membrane function are integrated, and FIG. 2C illustrates a process of bone regeneration using graft material and a barrier membrane separately or an integrated biomaterial in which a bone graft material and a barrier membrane function are integrated;

FIG. 3 illustrates differential scanning microscope images showing integrated biomaterials prepared according to Examples 1, 2, and 3, wherein arrows indicate a collagen layer;

FIG. 4 illustrates the results of observing the degree of degradation of an upper collagen layer of each of the integrated biomaterials of Examples 1, 2, and 3 by collagenase, wherein arrows indicate a collagen layer; and

FIG. 5 illustrates the results of observing the degree of bone regeneration of each of the integrated biomaterials of Examples 1, 2, and 3 after being implanted into bone defect sites of rabbits, wherein arrows indicate a collagen layer, G denotes a bone graft material, and NB denotes new bone.

### DETAILED DESCRIPTION AND EXEMPLARY EMBODIMENTS

Unless defined otherwise, all technical and scientific terms used herein have the same meaning as is commonly understood by one of ordinary skill in the art to which the present invention pertains. Generally, the nomenclature used herein is well known in the art and commonly used.

In the present invention, it was confirmed that, when an integrated biomaterial prepared by forming an upper layer formed of an extracellular matrix protein on a lower structure consisting of an extracellular matrix protein and a bone mineral is used as a bone graft material, the lower structure realizes a bone tissue environment to facilitate the regeneration of new bone, and the upper layer enables the bone graft material to be stably maintained on a bone defect site and realizes a natural bone tissue environment at a graft site by preventing the infiltration of epithelial tissue or connective tissue, thereby maximizing bone tissue regeneration capacity.

In addition, the integrated biomaterial may be prepared by inducing physical binding between the two structures in an initial preparation process without using an additional reagent such as a chemical crosslinking agent, other than main raw materials, and thus toxicity problems caused by a chemical crosslinking agent may be prevented, and an upper layer and a lower layer are not separated from each other even after hydration to thus also achieve structural stability.

In addition, compared with a case in which, when a lower structure and an upper layer are separately implanted, connective tissue infiltrates into a space between the two structures and thus interferes with a bone regeneration process, the integrated biomaterial has no space between the two structures, and thus connective tissue does not infiltrate into a side surface thereof so that the bone regeneration process proceeding from the bottom thereof is smoothly and effectively induced (see FIG. 2C).

Therefore, an embodiment of the present invention relates to an integrated biomaterial for bone tissue regeneration comprising a lower structure including an extracellular matrix protein and a bone mineral component and an upper layer including an extracellular matrix protein.

Another embodiment of the present invention relates to a use of an integrated biomaterial for bone tissue regeneration, the integrated biomaterial comprising: a lower structure including an extracellular matrix protein and a bone mineral component; and an upper layer including an extracellular matrix protein.

Another embodiment of the present invention relates to a method of regenerating bone tissue, comprising implanting, into an individual in need of bone tissue regeneration, an integrated biomaterial comprising: a lower structure including an extracellular matrix protein and a bone mineral component; and an upper layer including an extracellular matrix protein.

Another embodiment of the present invention relates to a method of preparing the integrated biomaterial for bone tissue regeneration, comprising: (a) molding a lower structure mixture including an extracellular matrix protein and bone mineral particles; (b) aligning a structure of the lower structure mixture including an extracellular matrix protein and bone mineral particles; (c) placing an upper layer including an extracellular matrix protein thereon; (d) binding the upper layer and the lower structure; (e) lyophilizing the resulting structure; and (f) thermally cross-linking the extracellular matrix protein of the upper layer.

In the present invention, the upper layer including an extracellular matrix protein must be organically bound to the lower structure including an extracellular matrix protein and a bone mineral component, which is a bone tissue-like biomaterial, and must not be separated therefrom when applied in vivo. In addition, since the upper layer including an extracellular matrix protein must be maintained for at least one week when implanted in vivo, the degree of degradation by a protease such as collagenase should be 10% (w/w) or less with respect to the total weight. In the present invention, in all of the integrated biomaterials prepared according to Examples 1 to 3, the collagen degradation rate of the upper layer by collagenase was in the range of 2.41% (w/w) to 5.90% (w/w) at the point of two weeks after collagen degradation, from which it was confirmed that the retention of the upper layer as a barrier membrane could last one week or longer.

In process (a) of the present invention, a mold is filled with a lower structure mixture including an extracellular matrix protein and bone mineral particles and molded.

In process (b) of the present invention, the alignment of the structure of the lower structure mixture of an extracellular matrix protein and bone mineral particles means that hydrophobic bonds, hydrogen bonds, or the like are formed between protein chains as a distance between the protein chains becomes narrow, thereby aligning protein chain arrangement, resulting in structural stabilization.

The concentration of the extracellular matrix protein used in process (c) of the present invention ranges from 0.5% (w/w) to 10% (w/w), more preferably in the range of 2% (w/w) to 5% (w/w), with respect to a total concentration of the biomaterial.

Process (d) of the present invention is a process of binding the upper layer (i.e., an extracellular matrix protein layer) and the lower structure (i.e., a mixture including an extracellular matrix protein and bone mineral particles) through gelation using a strong base, and the strong base may be selected from the group consisting of sodium hydroxide, potassium hydroxide, calcium hydroxide, barium hydroxide, ammonium hydroxide, calcium carbonate, potassium carbonate, and ammonia, but the present invention is not limited thereto.

Process (e) of the present invention may be performed by freezing at −1.5° C. for 2 hours or longer, followed by freezing at −20° C. at a freezing rate of 1° C./min, but a lyophilization method commonly used in the art may be applied.

Process (f) of the present invention is intended to extend the degradation time by dehydrothermal treatment of the extracellular matrix protein of the upper layer, and thermal crosslinking may be performed by treatment thereof at 140° C. to 160° C. for 48 hours to 168 hours.

Meanwhile, the method of preparing the integrated biomaterial for bone tissue regeneration may further include, after process (e), (g) adding an antimicrobial or anti-inflammatory functional material; and (h) lyophilization.

In the present invention, the extracellular matrix protein of the lower structure and the upper layer may be one or more selected from the group consisting of collagen, hyaluronic acid, elastin, chondroitin sulfate, and fibroin. As such an extracellular matrix, one derived from a human or an animal or any recombinant protein produced from a microorganism may be used. In particular, in the case of collagen, it is preferable to use type 1 or type 3 isolated from pig skin.

In the present invention, the bone mineral component may be one or more selected from the group consisting of living organism-derived bone mineral powder which originate from allogenic bone or xenogenic bone, synthetic hydroxyapatite, and tricalcium phosphate micropowder.

In the present invention, a ratio of the bone mineral component to the extracellular matrix protein may be varied, and the content of the bone mineral component is preferably 80 wt % or more, more preferably in the range of 80 wt % to 95 wt %, with respect to the total weight of the integrated biomaterial. A total content of the extracellular matrix protein of the lower structure and the extracellular matrix protein of the upper layer is preferably 5 wt % or more, more preferably in the range of 5 wt % to 20 wt %, with respect to the total weight of the integrated biomaterial.

Meanwhile, an amount ratio (weight ratio) of the extracellular matrix protein of the upper layer to the extracellular matrix protein of the lower structure preferably ranges from 0.13-1.3:1, and particularly, when the content ratio (weight ratio) of the extracellular matrix protein of the upper layer to the extracellular matrix protein of the lower structure is 0.5:1, it is the most preferable in terms of a significant increase in new bone formability. In this case, when the content of the extracellular matrix protein of the upper layer is less than 0.13 parts by weight with respect to 1 part by weight of the extracellular matrix protein of the lower structure, the upper layer is too thin (about 200 μm or less), and thus is unable to function as a barrier membrane for preventing infiltration of connective tissue, and accordingly, this case is not suitable for use as an integrated biomaterial for bone tissue regeneration. When the content of the extracellular matrix protein of the upper layer is greater than 1.3 parts by weight with respect to 1 part by weight of the extracellular matrix protein of the lower structure, the concentration of the extracellular matrix protein of the upper layer is too higher than that of the extracellular matrix protein in the lower structure, thus exhibiting higher density, and thus in the processes of placing the upper layer and binding the upper layer to the lower structure, an interface between the upper layer and the lower structure becomes unclear due to the density difference, and the bone mineral included in the lower structure is introduced into the upper layer such that an extracellular matrix protein layer of the upper layer is unable to properly act as a barrier membrane, and thus this case is not suitable for use as an integrated biomaterial for bone tissue regeneration.

In the present invention, the upper layer including an extracellular matrix protein preferably has a thickness of 20% to 35% of the entire biomaterial thickness. Preferably, the thickness of the upper layer is in the range of 0.5 mm to 1.5 mm and the thickness of the lower structure is in the range of 1 mm to 6 mm. More preferably, the thickness of the upper layer may be 1 mm and the thickness of the lower structure may range from 2 mm to 4 mm, but the present invention is not limited thereto.

The integrated biomaterial according to the present invention may further comprise an antimicrobial or anti-inflammatory functional material, and the antimicrobial or anti-inflammatory functional material may be, but is not limited to, any one or more selected from the group consisting of an antimicrobial agent, an antibiotic, and a peptide or protein with an anti-inflammatory function.

In the present invention, the antimicrobial agent may be, but is not limited to, sodium ethylenediaminetetraacetate, sodium copper chlorophyllin, a synthetic material containing fluorine or chlorine such as sodium fluoride or benzethonium chloride, aromatic carboxylic acid including benzoic acid and the like, allantoin, or tocopherol acetate.

In the present invention, the antibiotic may be, but is not limited to, minocycline, tetracycline, doxycycline, chlorohexidine, ofloxacin, tinidazole, ketoconazole, or metronidazole.

The antimicrobial peptide may be a peptide derived from human β-defensin, and the antimicrobial peptide may be selected from peptides having the amino acid sequences of SEQ ID NOS: 1 to 3, but the present invention is not limited thereto.

SEQ ID NO: 1 (BD3-3):
G-K-C-S-T-R-G-R-K-C-C-R-R-K-K
SEQ ID NO: 2 (BD3-3-M1):
G-K-C-S-T-R-G-R-K-C-M-R-R-K-K
SEQ ID NO: 3 (BD3-3-M2):
G-K-C-S-T-R-G-R-K-M-C-R-R-K-K

### EXAMPLES

Hereinafter, the present invention will be described in further detail with reference to the following examples. These examples are provided for illustrative purposes only, and it will be obvious to those of ordinary skill in the art that the scope of the present invention is not construed as being limited by these examples. Thus, the substantial scope of the present invention should be defined by the appended claims and equivalents thereto.

### Example 1: Integrated Biomaterial in which Lower Structure Consisting of Bovine Bone-Derived Bone Mineral Particles and Collagen, and Collagen Upper Layer are Integrated (Integrated Biomaterial: 7.7% Collagen Contained, Collagen Concentration of Upper Layer: 0.5%)

Bovine bone-derived bone mineral particles were prepared to have a particle size of 0.4 mm to 0.8 mm. 27 g of a bone mineral component was mixed with 50 mL of 4.0% (w/v) pig skin-derived collagen (2 g collagen) solution dissolved in 0.5 M acetic acid, and a mold was filled with the resulting mixture, and the lower structure was aligned on a clean bench. Separately, 50 mL of a 0.5% (w/v) pig skin-derived collagen (0.25 g collagen) solution dissolved in 0.5 M acetic acid was dispensed on the mixture to form an upper collagen layer. The weight of the used collagen was 7.7% (w/w) of the total weight. After the upper collagen layer was dispensed, the layer was left on a clean bench for a certain period of time. The resulting layer was left in ammonia vapor saturated with 25% to 30% aqueous ammonia for hours or longer to be gelled, and then washed with purified water to neutralize the pH.

After washing, the resulting product was frozen at −1.5° C. for 2 hours or more, and then frozen to −20° C. at a freezing rate of 1° C./min. After confirming that the integrated biomaterial was completely frozen at −20° C., lyophilization was performed for 48 hours. The lyophilized integrated biomaterial was cross-linked in a vacuum oven at 140° C. for 120 hours, thereby completing the preparation of an integrated biomaterial.

### Example 2: Integrated Biomaterial in which Lower Structure Consisting of Bovine Bone-Derived Bone Mineral Particles and Collagen, and Upper Collagen Layer are Integrated (Integrated Biomaterial: 10.0% Collagen Contained, Collagen Concentration of Upper Layer: 2.0%)

Bovine bone-derived bone mineral particles were prepared to have a particle size of 0.4 mm to 0.8 mm. 27 g of a bone mineral component was mixed with 50 mL of 4.0% (w/v) pig skin-derived collagen (2 g collagen) solution dissolved in 0.5 M acetic acid, and a mold was filled with the resulting mixture, and the lower structure was aligned on a clean bench. Separately, 50 mL of a 2.0% (w/v) pig skin-derived collagen (1 g collagen) solution dissolved in 0.5 M acetic acid was dispensed on the mixture to form an upper collagen layer. The weight of the used collagen was 10.0% (w/w) of the total weight. After the upper collagen layer was dispensed, the layer was left on a clean bench for a certain period of time. The resulting layer was left in ammonia vapor saturated with 25% to 30% aqueous ammonia for hours or longer to be gelled, and then washed with purified water to neutralize the pH. After washing, the resulting product was frozen at −1.5° C. for 2 hours or more, and then frozen to −20° C. at a freezing rate of 1° C./min. After confirming that the integrated biomaterial was completely frozen at −20° C., lyophilization was performed for 48 hours. The lyophilized integrated biomaterial was cross-linked in a vacuum oven at 140° C. for 120 hours, thereby completing the preparation of an integrated biomaterial.

### Example 3: Integrated Biomaterial in which Lower Structure Consisting of Bovine Bone-Derived Bone Mineral Particles and Collagen, and Upper Collagen Layer are Integrated (Integrated Biomaterial: 14.3% Collagen Contained, Collagen Concentration of Upper Layer: 5.0%)

Bovine bone-derived bone mineral particles were prepared to have a particle size of 0.4 mm to 0.8 mm. 27 g of a bone mineral component was mixed with 50 mL of 4.0% (w/v) pig skin-derived collagen (2 g collagen) solution dissolved in 0.5 M acetic acid, and a mold was filled with the resulting mixture, and the lower structure was aligned on a clean bench. Separately, 50 mL (2.5 g collagen) of a 5.0% (w/v) pig skin-derived collagen (2.5 g collagen) solution dissolved in 0.5 M acetic acid was dispensed on the mixture to form an upper collagen layer. The weight of the used collagen was 14.3% (w/w) of the total weight. After the upper collagen layer was dispensed, the layer was left on a clean bench for a certain period of time. The resulting layer was left in ammonia vapor saturated with 25% to 30% aqueous ammonia for 3 hours or longer to be gelled, and then washed with purified water to neutralize the pH. After washing, the resulting product was frozen at −1.5° C. for 2 hours or more, and then frozen to −20° C. at a freezing rate of 1° C./min. After confirming that the integrated biomaterial was completely frozen at −20° C., lyophilization was performed for 48 hours. The lyophilized integrated biomaterial was cross-linked in a vacuum oven at 140° C. for 120 hours, thereby completing the preparation of an integrated biomaterial.

### Comparative Example 1: Biomaterial Consisting of Lower Structure Only, which Consists of Bovine Bone-Derived Bone Mineral Particles and Collagen (Biomaterial: 6.90% Collagen Contained, Collagen Concentration of Upper Layer: 0%)

Bovine bone-derived bone mineral particles were prepared to have a particle size of 0.4 mm to 0.8 mm. 27 g of a bone mineral component was mixed with 50 mL of 4.0% (w/v) pig skin-derived collagen (2 g collagen) solution dissolved in 0.5 M acetic acid, and a molding was filled with the resulting mixture, and the lower structure was aligned on a clean bench. The weight of the used collagen was 6.90% of the total weight. After the upper collagen layer was dispensed, the layer was left on a clean bench for a certain period of time. The upper collagen layer was gelled by a strong base, and then washed with purified water to neutralize the pH. After washing, the resulting product was frozen at −1.5° C. for 2 hours or more, and then frozen to −20° C. at a freezing rate of 1° C./min. After confirming that the integrated biomaterial was completely frozen at −° C., lyophilization was performed for 48 hours. The lyophilized integrated biomaterial was cross-linked in a vacuum oven at 140° C. for 120 hours, thereby completing preparation of an integrated biomaterial. The amounts of a bone graft material and collagen used are shown in Table 1 below.

TABLE 1

| | | | | |
|---|---|---|---|---|
| Conditions | | | | Comparative |
| Used amount | Example 1 | Example 2 | Example 3 | Example 1 |

Amounts of bone graft material and collagen used in lower structure

| | | | | |
|---|---|---|---|---|
| Bone mineral | 27 | 27 | 27 | 27 |
| (g) | | | | |
| Collagen (g) | 2 | 2 | 2 | 2 |

Amount of collagen used in upper layer

| | | | | |
|---|---|---|---|---|
| Collagen (g) | 0.25 | 1 | 2.5 | 0 |
| Weight (%) of | 2.25/29.25*100 = | 3/30*100 = | 4.5/31.5*100 = | 2/29*100 = |
| collagen with | 7.69 (%) | 10.0 (%) | 14.28 (%) | 6.90 (%) |
| respect to | | | | |
| total amount | | | | |
| (Amount of collagen | (2 + 0.25)/(2.25 + 27)*100 = | (2 + 1)/(3 + 27)*100 = | (2 + 2.5)/(4.5 + 27)*100 = | (2 + 0)/(2 + 27)*100 = |
| used in upper and | 7.69 (%) | 10.0 (%) | 14.28 (%) | 6.90 (%) |
| lower)/(amounts of | | | | |
| collagen in upper | | | | |
| and lower and bone | | | | |
| graft material) | | | | |

### Experimental Example 1: Observation of Structure of Prepared Integrated Biomaterial

The integrated biomaterials prepared according to Examples 1 to 3 and our commercial product (OCS-B Xenomatrix Collagen, NIBEC, Korea) as a control were observed using a differential scanning electron microscope. Each tissue-structured mimetic was coated with platinum and observed with a field emission scanning electron microscope (FE-SEM, Jeol, S-4700, Japan).

FIG. 3 is a set of differential scanning microscope images of the integrated biomaterials of Examples 1 to 3. It was observed that the collagen and bone mineral components were uniformly mixed in the composite structures prepared in Examples 1 to 3, and it was confirmed that the lower structure consisting of bone mineral and collagen and the upper collagen layer were firmly bound to each other without an empty space therebetween.

### Experimental Example 2: Test for Degradation by Collagenase

20 μg/mL of collagenase (0.247 U/mg lyophilizate) was contained in an HBSS (Salt Solution) solution, and the integrated biomaterials of Examples 1 to 3 were left for a certain period of time and the degree of degradation thereof was examined.

$$( \% ) = \frac{ ( a - b )}{ ( a )} \times 100$$

a: weight before degradability test (g)

b: weight after degradability test (g)

* Since the bone graft material is not degraded by collagenase, it does not affect collagen degradation rate.

After 2 weeks, collagen degradation activity in the upper collagen layer and the lower structure (a bone graft material and collagen mixed), which are the whole structure, was tested, and the results showed degradation of a maximum of at most 6.90% and at least 1% with respect to weight before the test. Considering that the degradation rate of collagen in the lower structure was 1% in the control, the degradation rate of collagen only in the upper layer of each of Examples 1 to 3 was 2.41% to 5.90% respectively obtained by subtraction of 1%.

It was confirmed that the upper collagen layer was retained until 2 weeks, and in Examples 2 and 3, the degradation rate of the upper collagen layer was maintained at 3% or less.

The degradation degree according to the concentration of collagenase is shown in Table 2 below.

TABLE 2

| | | | | Collagen concentration of upper layer of integrated biomaterials according to examples | | | | | | | | |
|---|---|---|---|---|---|---|---|---|---|---|---|---|
| | Control | | | | | | | | | | | |
| | 0% (Comparative | | | 0.5% | | | 2% | | | 5% | | |
| | Example 1) | | | (Example 1) | | | (Example 2 ) | | | (Example 3) | | |
| | Weight according to condition | | | | | | | | | | | |
| | | Weight | | | Weight | | | Weight | | | Weight | |
| Concentration | Initial | after 2 | | Initial | after 2 | | Initial | after 2 | | Initial | after 2 | |
| of | weight | weeks | Degradation | weight | weeks | Degradation | weight | weeks | Degradation | weight | weeks | Degradation |
| collagenase | (a) | (b) | rate *1) | (a) | (b) | rate *1) | (a) | (b) | rate *1) | (a) | (b) | rate *1) |
| 20 | 1.0190 | 1.0088 | 1.00% | 1.0507 | 0.9782 | 6.90% | 0.8508 | 0.8218 | 3.41% | 0.8710 | 0.8400 | 3.56% |
| μg/ml | (g) | (g) | Degradation | (g) | (g) | Degradation | (g) | (g) | Degradation | (g) | (g) | Degradation |
| | | | rate *2) | | | rate *2) | | | rate *2) | | | rate *2) |
| | | | | | | 5.90% | | | 2.41% | | | 2.56% |

*1) Degradation rate of collagen in integrated structure (including both the upper collagen layer and the lower structure)
*2) Degradation rate of collagen used in preparation of upper collagen layer

FIG. 4 illustrates differential scanning electron microscope images showing surfaces of the integrated biomaterials of Examples 1 to 3 and control, wherein the images were acquired after the degradation test was completed to identify the degree of degradation of collagen in the upper layer.

### Experimental Example 3: Test for Bone Regeneration in Animal

An 8 mm defect was formed in the skull of rabbits, and each of the integrated biomaterials of Examples 1 to 3 and the biomaterial of Comparative Example 1 as a control were implanted thereinto for 8 weeks, and then the degree of formation of new bone and the regeneration capacity of surrounding tissues were observed (see FIG. 5).

When the biomaterial of Example 2 was implanted, more new bone was formed than the other groups. In addition, the integrated biomaterial of Example 2 showed an increase in the area of a new bone by about 50% compared to Comparative Example 1. The histomorphometry results thereof are shown in Table 3 below.

TABLE 3

| | New bone | Connective tissue | Bone graft |
|---|---|---|---|
| Groups | area (%) | area (%) | area (%) |
| Comparative | 10.81 ± 8.49 | 62.35 ± 11.26 | 26.84 ± 8.92 |
| Example 1 | | | |
| Example 1 | 12.85 ± 7.63 | 65.14 ± 12.35 | 22.01 ± 7.21 |
| Example 2 | 20.13 ± 6.63 | 56.95 ± 7.96 | 22.92 ± 9.49 |
| Example 3 | 16.58 ± 10.52 | 58.54 ± 11.32 | 24.88 ± 9.79 |

While the present invention has been particularly described with reference to specific embodiments thereof, it will be obvious to those of ordinary skill in the art that these exemplary embodiments are provided for illustrative purposes only and are not intended to limit the scope of the present invention. Therefore, the substantial scope of the present invention should be defined by the appended claims and equivalents thereto.

### INDUSTRIAL APPLICABILITY

In an integrated biomaterial for bone tissue regeneration according to the present invention, a lower structure consisting of extracellular matrix protein and bone mineral components realizes a natural bone tissue environment, and thus facilitates the regeneration of a new bone, and particularly, an upper layer consisting of an extracellular matrix protein is placed at an appropriate ratio, and thus not only prevents the infiltration of epithelial tissue or connective tissue but also maximizes bone tissue regeneration capability.

### Sequence List Free Text

Electronic file attached

1. An integrated biomaterial for bone tissue regeneration, the integrated biomaterial comprising: a lower structure comprising an extracellular matrix protein and a bone mineral component; and an upper layer comprising an extracellular matrix protein.

2. The integrated biomaterial according to claim 1, wherein the extracellular matrix protein of each of the lower structure and the upper layer comprises any one or more selected from the group consisting of collagen, hyaluronic acid, elastin, chondroitin sulfate, and fibroin.

3. The integrated biomaterial according to claim 1, wherein the bone mineral component comprises one or more selected from the group consisting of a living organism-derived bone mineral powder originating from allogenic bone or xenogenic bone, synthetic hydroxyapatite, and tricalcium phosphate micropowder.

4. The integrated biomaterial according to claim 1, wherein a content of the bone mineral component ranges from 80 wt % to 95 wt % with respect to a total weight of the integrated biomaterial.

5. The integrated biomaterial according to claim 1, wherein a total content of the extracellular matrix protein of the lower structure and the extracellular matrix protein of the upper layer ranges from 5 wt % to 20 wt % with respect to a total weight of the integrated biomaterial.

6. The integrated biomaterial according to claim 1, wherein an amount ratio (weight ratio) of the extracellular matrix protein of the upper layer to the extracellular matrix protein of the lower structure ranges from 0.13-1.3:1.

7. The integrated biomaterial according to claim 1, further comprising an antimicrobial or anti-inflammatory functional material.

8. A method of preparing an integrated biomaterial for bone tissue regeneration, the method comprising the following processes:
(a) molding a lower structure mixture comprising an extracellular matrix protein and bone mineral particles;
(b) aligning a structure of the lower structure mixture comprising an extracellular matrix protein and bone mineral particles;
(c) placing an upper layer comprising an extracellular matrix protein thereon;
(d) binding the upper layer and the lower structure;
(e) lyophilizing the resulting structure; and
(f) thermally cross-linking the extracellular matrix protein of the upper layer.

9. The method according to claim 8, wherein the extracellular matrix protein comprises any one or more selected from the group consisting of collagen, hyaluronic acid, elastin, chondroitin sulfate, and fibroin.

10. The method according to claim 8, wherein the bone mineral component comprises one or more selected from the group consisting of a living organism-derived bone mineral powder originating from allogenic bone or xenogenic bone, synthetic hydroxyapatite, and tricalcium phosphate micropowder.

11. The method according to claim 8, wherein a content of the bone mineral component ranges from 80 wt % to 95 wt % with respect to a total weight of the integrated biomaterial.

12. The method according to claim 8, wherein a total content of the extracellular matrix protein of the lower structure and the extracellular matrix protein of the upper layer ranges from 5 wt % to 20 wt % with respect to a total weight of the integrated biomaterial.

13. The method according to claim 8, wherein an amount ratio (weight ratio) of the extracellular matrix protein of the upper layer to the extracellular matrix protein of the lower structure ranges from 0.13-1.3:1.

14. The method according to claim 8, wherein the upper layer and the lower structure of process (d) are bound through gelation using a strong base.

15. The method according to claim 8, wherein process (e) is performed by thermal crosslinking at 140° C. to 160° C. for 48 hours to 168 hours.

16. The method according to claim 8, further comprising, after process (e):
(g) adding an antimicrobial or anti-inflammatory functional material; and
(h) lyophilizing the resulting structure.

17. The method according to claim 16, wherein the antimicrobial or anti-inflammatory functional material comprises any one or more selected from the group consisting of an antimicrobial agent, an antibiotic, and a peptide or protein with an anti-inflammatory function.

18. The method according to claim 17, wherein the antimicrobial agent is chlorohexidine.
